(19) United States
(12) Patent Application Publication
(10) Pub. No.: US 2013/0316381 A1
(43) Pub. Date: Nov. 28, 2013

(54) METHOD AND REAGENT DEVICE FOR DETERMINING ANTI-RA33 ANTIBODY IGG

(71) Applicant: Deming Hu, Shenzhen (CN)

(72) Inventor: Deming Hu, Shenzhen (CN)

(73) Assignee: SHENZHEN YHLO BIOTECH CO., LTD., Shenzhen, Guangdong (CN)

(21) Appl. No.: 13/978,233

(22) PCT Filed: Jan. 4, 2011
(86) PCT No.: PCT/CN11/70028

§ 371 Date: Aug. 5, 2013

(51) Int. Cl. G01N 33/543

(52) U.S. Cl. CPC G01N 33/54306
USPC 435/7.92; 435/287.2

(57) ABSTRACT

The present invention provides a method for determining anti-RA33 antibody by ELISA based on a special device. The device comprises a substrate, wherein 8 wells are formed on the substrate, and one end of the substrate is provided with a handle and the handle is adhered with a bar-shaped code with the information of detecting reagent. By using the method and device of present invention, detection reagents can be placed in a single analysis device, thus the operation is simple and it is not easy to go wrong so that the correctness of the detection result is ensured.

### FIELD OF THE PRESENT INVENTION

The present invention relates to a method for determining an anti-RA33 antibody IgG and a reagent device, which belongs to the technical field of biological detection.

### BACKGROUND OF THE RELATED ART

RA (Rheumatoid Arthritis) is a most common systemic autoimmune disease, which is mainly represented as chronic, symmetrical, arthritis erosive synovitis; wherein the disease is mostly progressive; and about one third of the patients may be crippled if the disease may not be treated timely. Therefore, early diagnosis and early treatment are the keys to treat Rheumatoid Arthritis. The diagnostic criteria amended by ACR (American College of Rheumatology) in 1987 is based on clinical manifestation; and the specificity of rheumatoid factors (RF) which serves as the unique diagnostic criteria is poor. Therefore, rheumatologists are all committed to search biological markers for the early diagnosis of RA (Rheumatoid Arthritis) and have gained certain achievements.

An anti-RA33 antibody was reported by Hassfeld et al. for the first time in 1989; as the antibody is a more specific antibody for diagnosing RA (Rheumatoid Arthritis) and reacts with nucleic acid protein with a molecular weight of 33 KD, the antibody is accordingly named as anti-RA33 antibody. Target antigen of the anti-RA33 antibody is nucleic acid binding protein with a molecular weight of 33 KD, which is consistent with A2 protein in hnRNP; and the antigen is from HeLa cell or Ehrilich cell. IBT (Immunoblotting Test) is adopted as a detection method. At the same time of detecting the anti-RA33 antibody in 1992, Hassfeld found that partial RA patients also had an anti-36 KD nucleic acid protein antibody; moreover, the anti-36 KD nucleic acid protein antibody is irrelevant to an anti-proliferating cell nuclear antigen antibody (Anti-PCNA Antibody). The anti-RA33 antibody and an anti-RA36 antibody always appear at the same time; moreover, two characteristic bands formed at positions of 33 KD and 36 KD which exist at the same time during the IBT (Immunoblotting Test) has specificity on diagnosing RA. In various indexes of early RA diagnosis, the specificity of the anti-RA33 antibody is highest; the positive rate of the antibody in RA is 35.8 percent and the antibody appears in early RA in particular. The succession of the anti-RA33 antibody is irrelevant to the disease and the drugs used.

In addition, the anti-RA33 antibody except exists in the bodies of partial RA patients, the anti-RA33 antibody also exists in about 20 percent of SLE (Systemic Lupus Erythematosus) patients and 40 percent to 60 percent of MCTD (Mixed Connective Tissue Disease) patients. However, it is reported in literatures that the anti-RA33 antibody in SLE patients and MCTD patients always exist at the same time with another two autoantibodies, which include anti-U1-RNP antibody and anti-Sm antibody; therefore, SLE (Systemic Lupus Erythematosus) and MCTD (Mixed Connective Tissue Disease) may be excluded by examining the anti-U1-RNP antibody and the anti-Sm antibody at the same time.

IBT (Immunoblotting Test) is earliest and most frequently used for detecting the anti-RA33 antibody; in addition, ELISA (Enzyme Linked Immunosorbent Assay) may also be used for detecting; however, these method both have disadvantages.

### I. IBT (Immunoblotting Test):

IBT (Immunoblotting Test) was reported by Hassfeld et al. for the first time in 1989 to detect the anti-RA33 antibody in serum of RA patients. IBT (Immunoblotting Test) is hybridization

Technique which combines high resolution gel electrophoresis technique with an immunochemistry analysis technique. IBT (Immunoblotting Test), which has the advantages of large analysis capacity, high sensitivity and strong specificity, etc, is a most common method for detecting protein characteristics, expression and distribution, such as qualitative and quantitative detection of tissue antigens, quantity determination of peptide molecules as well as virus antibody or antigen detection, and the like. The advantages of IBT (Immunoblotting Test) are as follows:
(1) Only qualitative and semiquantitative analysis may be carried out; specific quantity of a matter detected may not be obtained.
(2) The operating steps are fussy and the test time is longer; moreover, it is more difficult to develop this detection in grass-roots;
(3) The detection sensitivity and specificity are to be improved.
(4) Meanwhile, IBT (Immunoblotting Test) is not beneficial for clinic popularization and application as the whole process of extracting the antigens is complex, the time is lone, the requirements on instruments is higher, and the antigens are difficult to keep for a long period of time.

### II. ELISA (Enzyme Linked Immunosorbent Assay)

Compared with other biological detection or immunological detection, application of ELISA (Enzyme Linked Immunosorbent Assay) is limited due to a pluralities of disadvantages thereof on detection method, technique and tool or product, which mainly comprise the several aspects as follows:
(1) 12×8 type, 8×12 type or entire-plate type 96 hole special microplates are used as antigen or antibody coating articles and reaction vessels, which may only be used by dividing into 12 batches, 8 batches or entirely used for once.
(2) 11 reagents are used for quantitative determination, wherein each reagent needs to be held by a reagent bottle and each reagent needs to be respectively filled into the micropores of the microplate by replacing a new solution nozzle; not only the types of the reagent bottles are more, but also the operation of filling the reagent is fussy; if a full-automatic enzyme immunoassay analyzer is not used, then all the operations need to be carried out manually; however, the price of the full-automatic enzyme immunoassay analyzer is very expensive, which causes larger investment.
(3) No bar codes of reagent information for each detection or detections in each time are provided; therefore, the batch number and validity information of the detection reagent may only be known by checking a label of an external packaging box for a kit; moreover, the known information is not controlled during a detection process, which has high randomness.
(4) The detection reagents during the detection process are open, which are easy to cause cross pollution between the reagents so as to affect a detection result.
(5) The detection process is manually operated when the full-automatic enzyme immunoassay analyzer is not used for detecting, which may cause not accurate dosage of reagent or sample; moreover, the operating process is extremely fussy and complex, which is easy to cause operating errors as well as poor accuracy and precision of the detection result.
(6) Both 96 portions of completed set of reagents are needed both for configuration and use for a detection item; if 10 items are needed to detect, then 960 portions of reagents needs to be configured and used; if 10 different items of one sample need to be detected only, 960 portions of reagents also need to be configured.

### SUMMARY OF THE PRESENT INVENTION

In order to solve the defects of the existing anti-RA33 antibody IgG analysis and detection method, the present invention provides a new detection method, a reagent device and ancillary reagents. The method is based on an ELISA (Enzyme Linked Immunosorbent Assay) technique to search a set of simpler, more accurate and effective method and reagents to implement quantitative determination so as to meet the demands of clinical diagnosis.

The method is based on an ELISA (Enzyme Linked Immunosorbent Assay) principle to implement immunological detection on the anti-RA33 antibody IgG, and relates to an independent, single portion and one-off analysis method, a reagent device and ancillary reagents used for detecting the anti-RA33 antibody IgG by means of ELISA (Enzyme Linked Immunosorbent Assay), wherein a plurality of reagents needed for detecting the anti-RA33 antibody IgG by means of ELISA (Enzyme Linked Immunosorbent Assay) may be held on one analytical device; relevant immunological detections may be conveniently carried out in accordance with use demands of the detection items through the method, thus providing better basis for clinical application.

The present invention provides a method for determining an anti-RA33 antibody IgG, wherein the method is implemented by a to a kit consisting of an ELISA (Enzyme-linked Immunosorbent Assay) analytical reagent device and ancillary reagents, wherein the analytical reagent device comprises a substrate provided with 8 holes, and a handle located at one end of the substrate; various specific reagent solutions between the holes of the reagent device are filled and aspirated by a specific analytical instrument to make samples react with the reagents, then a numerical value of solution color after reaction is measured, and finally the measured numerical value is processed to obtain a detection result.

According to the method for determining the anti-RA33 antibody IgG, an anti-RA33 antibody IgG to be detected in a sample to be detected is reacted with a recombinant RA33 antigen to form a first immune complex; the first immune complex is reacted with a second antibody labeled by enzyme to form a second immune complex; chromogenic contrast analysis is carried out between the second immune complex formed by reaction and a chromogenic substrate so as to obtain contents of the anti-RA33 antibody IgG to be detected.

According to the method for determining the anti-RA33 antibody IgG, the second antibody is an anti-human IgG antibody labeled by HRP (Horse Radish Peroxidase).

The present invention provides a reagent device for determining an anti-RA33 antibody IgG, wherein the reagent device is provided with a substrate with 8 holes, a handle located at one end of the substrate, an analytical reagent device used for detecting the anti-RA33 antibody IgG by means of ELISA (Enzyme-linked Immunosorbent Assay) as well as components of corresponding quantities such as ancillary reagent calibrators, quality control materials and buffer washing solutions.

According to the reagent device for determining an anti-RA33 antibody IgG, a label of a detection reagent bar code is stuck on the handle located at one end of the substrate; numerical values of the bar code comprises such information as detection item code, detection reagent batch number, reagent validity, qualitative adjusted value/quantitative assay standard curve parameters, ELASA (Enzyme Linked Immunosorbent Assay) reaction type as well as serial numbers of reagents and analytical device corresponding to each detection.

According to the reagent device for determining an anti-RA33 antibody IgG, the holes comprises a reaction hole, a sample hole, a dilution hole and five reagent holes, wherein,
1) The sample hole is used for holding a solution to be detected.
2) The dilution hole is used for diluting samples.
3) The reaction hole is flat in the bottom and has very high light source/light path permeability; when a colourless/blank reagent solution is held, an absorbance value to visible light/ultraviolet light/fluorescent light is approaching to zero; a recombinant RA33 antigen required for detecting the anti-RA33 antibody IgG is coated in the reaction hole; the reaction hole is used for holding detection samples and detection reagents, and generates ELASA (Enzyme Linked Immunosorbent Assay) reaction with the detection samples and the detection reagents; moreover, the reaction hole is a vessel for ELASA (Enzyme Linked Immunosorbent Assay) reaction as well as color display and detection.
4) Each reagent hole is filled with a reagent required for detecting the anti-RA33 antibody IgG by means of ELASA (Enzyme Linked Immunosorbent Assay); after the reagent is filled, an opening edge of a micropore is closed.

According to the reagent device for determining an anti-RA33 antibody IgG, the reagents required for detecting the anti-RA33 antibody IgG by means of ELASA (Enzyme Linked Immunosorbent Assay) and held in the reagent holes comprise immune reaction inhibitor/neutralizer/blocker/absorbent, enzyme-conjugate solution, chromogenic substrate solution, chromogenic stop solution, reaction enhancer/accelerator and sample dilution solution required by the ELASA (Enzyme Linked Immunosorbent Assay) for detecting the anti-RA33 antibody IgG.

According to the reagent device for determining an anti-RA33 antibody IgG, the section shapes of the sample hole, the reaction hole, the dilution hole and the reagent holes comprise flat shape, V shape or U shape, or any combination between flat shape, V shape and U shape.

The method for determining an anti-RA33 antibody IgG comprises the steps as follows:
1) Starting up: after an instrument switch is turned on, the instrument may automatically carry out a series of examinations, so as to prepare for the normal operation of the instrument.
2) Preparation of examination programs: prepare corresponding solutions as required, comprising buffer washing solution, cleaning solution, disinfectant and distilled water or deionized water, and fill the solutions into corresponding solution tanks after completing the preparation.
3) Flushing examination
4) Preheating: after starting up, the instrument may enable a heating program to adjust temperature to the temperature to be detected.
5) Connected with a host machine: the instrument may be connected with the host machine through a RS232 serial port, thus submitting a result obtained by normal work of the instrument to a centralized system for processing.
6) Detection of anti-RA33 antibody IgG: the detection further comprises the steps as follows:
   i. Opening a packaging bag from one side with a sealing opening, selecting analytical devices with a quantity as required, deaerating and then sealing the opening of the packaging bag.
   ii. Examining the substrate in the reagent hole of the analytical device, wherein the substrate shall have no color change; otherwise, the substrate shall be discarded.
   iii. Respectively adding 50-100 Mu L undiluted samples into the sample holes of each analytical device, wherein for each batch of reagents changed, one analytical device and calibrator thereof shall be selected for calibrating the instrument.
   iv. Arranging the analytical devices into corresponding analytical device trays in the instruments and carrying out calibration and detection in accordance with an operation instruction.
   v. Arranging the analytical devices with corresponding quantity into the analytical device trays in accordance with the quantity required for detection, and arranging the analytical devices comprising the calibrators and the quality control materials before a detection position, wherein the instrument may automatically identify the bar codes of the analytical devices, the bar codes of the quality control materials and the bar codes of the calibrators; moreover, a selection line may be located at a ‘sample’ column or ‘detection’ column.
   vi. Clicking start to operate a detection list, scanning the bar codes of each analytical device, and numbering the quality control materials, the calibrators and the detected samples.
   vii. Operating the detection list, wherein the instrument may automatically operate according to bar code information; in accordance with the bar codes, the instrument may select correspondingly set standard curves; a program firstly detects the calibrator so as to calibrate a curve preset in the instrument; secondarily, the quality control materials are detected; if a detection result is within a marked scope, then the curve internally arranged is qualified, and may be used for detecting the samples; and finally, a sample detection program is started.
   viii. Dilution: a filling needle may automatically absorb the samples from the sample hole, pierces a hole sealing film to automatically absorb the dilution solution to dilute the samples in the dilution hole; after the action is completed, the diluted samples may be moved to the reagent hole by the filling hole and reacted for a period of time set by the program, and then the solution is removed.
   ix. Washing: the filling needle may absorb a certain quantity of washing solution from a corresponding solution tank to wash the reagent hole for three to five times, and then the solution is removed.
   x. The filling needle pierces a reagent hole sealing film to absorb a certain quantity of anti-human IgG antibody labeled by HRP (Horse Radish Peroxidase) to the reagent hole to react for a period of time set by the program, and then the solution is removed.
   xi. Repeating washing of step (9).
   xii. The filling needle pierces the sealing film of the reagent hole to absorb a certain quantity of enzyme reaction substrates to the reagent hole to react for a period of time set by the program.
   xiii. The filling needle pierces the sealing film of the reagent hole to absorb a certain quantity of stop solution and fill the solution into the reagent hole, and reads OD value in 10 minutes at 450 nm; if a dual wavelength method is selected for determination, then a reference wavelength is 620 nm to 690 nm.
7) Detection result: when a detection program operates completely, click data transmission with the host machine, the instrument may automatically send the result obtained by the normal work to the host machine and handle over to external data processing software to analyze and finally generate a report for consulting.
8) Shutdown: after the detection is completed, washing circulation shall be enabled before shutdown of the instrument; in this way, residual saline matter from solutions may be avoided from crystallizing in a solution path to prevent the instrument from being damaged or causing invalid detection result; after the washing is completed, the instrument automatically turns off a power supply.

The analytical reagent device relates to an independent, single purpose and one-off ELISA (Enzyme-linked Immunosorbent Assay) analytical reagent device for determining the anti-RA33 antibody IgG, and specially used for a specific analytical instrument.

According to the method of the present invention for determining the anti-RA33 antibody IgG and ancillary reagents by means of ELISA (Enzyme-linked Immunosorbent Assay), the analytical

Reagent device for implementing detection of the anti-RA33 antibody IgG relates to an independent, single purpose and one-off ELISA (Enzyme-linked Immunosorbent Assay) analytical reagent device specially used for a specific analytical instrument.

The method and the device of the present invention for determining the anti-RA33 antibody IgG inherit such characteristics of other detection methods as strong specificity, high sensitivity, good accuracy, low cost, low use requirement, simple and convenient operation, short detection result obtaining time and broad application, and solve a plurality of defects of other detection methods, which comprise the several aspects as follows specifically:
1. The determination method applies the ELISA (Enzyme-linked Immunosorbent Assay) principle, utilizes the specific analytical instrument, adopts the ancillary kit and the analytical reagent device to automatically implement qualitative/quantitative determination of the anti-RA33 antibody IgG, and relates to a brand new, applicable, practical, effective and rapid scheme for determining the anti-RA33 antibody IgG.
2. Independent and single portion detection reagent and analytical device are used, which does not need to use 12×8 type, 8×12 type or entire-plate type 96 hole special ELISA (Enzyme-linked Immunosorbent Assay) microplates as antigen or antibody coating articles and reaction vessels; when in use, only one portion of samples is needed to detect corresponding items, thus causing no wastes of reagents. If the quantity of the samples exceed one portion, the reagent and the analytical device may be used according to the actual quantity of the samples.
3. No matter qualitative determination or quantitative determination, the reagents required for each determination are held in the reagent holes of one analytical reagent device rather than held in different reagent bottles; in this way, not only the operation is extremely simple and convenient, but also operating errors are difficult to cause, thus guaranteeing the accuracy of a determination result.
4. Each analytical reagent device is equipped with a special bar code, wherein numerical values of the bar code comprise such information as detection item code, detection reagent batch number, reagent validity, quantitative assay standard curve parameters, detailed ELASA (Enzyme Linked Immunosorbent Assay) reaction type as well as serial numbers of reagents and analytical devices corresponding to detection, which shall not be altered randomly and are strictly controlled when in use; and in particular, when the detection reagents expiring the validities are used, the reagents are to be identified and a detection report is prevented from issuing, thus guaranteeing the detection accuracy.
5. Each detection reagent is effectively isolated and sealed, which may not cause cross pollution between the reagents so as to affect the detection result.
6. The device relates to an analytical reagent device specially used for the specific analytical instrument; during a detection process, a full-automatic precise filler is used for filling the detection reagents or samples, which has the advantages of automatic operation, precise dosage as well as high accuracy and precision of the detection result.
7. The completed set of reagents for the detection items are prepared according to the practical use demands; and in particular to multi-item detection, the configuration is more proper and situations of exceeding configuration and use may not occur.

### BRIEF DESCRIPTIONS OF THE DRAWINGS

FIG. 1 illustrates a section structure diagram of one example embodiment of a reagent device for determining an anti-RA33 antibody IgG according to the present invention;

FIG. 2 illustrates a plane vertical view of one example embodiment of the reagent device for determining an anti-RA33 antibody IgG according to the present invention;

FIG. 3 illustrates a section structure diagram of another example embodiment of a reagent device for determining an anti-RA33 antibody IgG according to the present invention;

FIG. 4 illustrates a plane vertical view of another example embodiment of the reagent device for determining an anti-RA33 antibody IgG according to the present invention;

FIG. 5 and FIG. 6 illustrate a section structure diagram of other example embodiment of a reagent device for determining an anti-RA33 antibody IgG according to the present invention; wherein,

1 refers to the sample hole; 2, 3, 4, 5 and 6 refer to the reagent holes; 7 refers to the reaction hole, 8 refers to the dilution hole; 9 refers to the handle; 10 refers to the sealing film; 11 refers to the substrate and 90 refers to the label.

### DETAILED DESCRIPTIONS OF THE EXAMPLE EMBODIMENTS

The following descriptions are further described by reference to the specific determination device and implementation steps of the determination method and the reagent device of the present invention, to make the public better understand the technical scheme of the present invention, rather than to limit the technical scheme. In fact, improvement on the steps of the method as well as increasing and decreasing, replacement and improvement on the structures of the corresponding reagent devices shall all fall within the protection scope of the present invention.

### Example Embodiment 1 Indirect ELISA (Enzyme-linked Immunosorbent Assay) Method for Determining Anti-RA33 Antibody IgG and Kit as Well as Reagent Device

The present invention relates to a set of simpler, more accurate and effective method based on ELISA (Enzyme-linked Immunosorbent Assay) technique, and adopts a basic principle of indirect ELISA (Enzyme-linked Immunosorbent Assay). A recombinant RA33 antigen is absorbed on a solid phase; a specific antibody in diluted human serum is combined with the antigen by incubation, and then the antibody not combined with the solid phase is removed by washing; anti-human IgG enzyme conjugate labeled by HRP (Horse Radish Peroxidase) is added for incubation. The enzyme conjugate which is not combined is removed, and enzyme chromogenic substrate is added. A color generated is proportional to the concentrate of the specific antibody in the sample to be detected. The method mainly implements the immunological detection of the anti-RA33 antibody IgG through the analytical reagent device and the ancillary reagents used for ELISA (Enzyme-linked Immunosorbent Assay). Judgment may be made quickly and accurately for the demand of clinical diagnosis by ELISA (Enzyme-linked Immunosorbent Assay) as well as application of the analytical reagent device and the reagents of the specific analytical instrument. The detailed structure of the analytical device thereof is as follows:

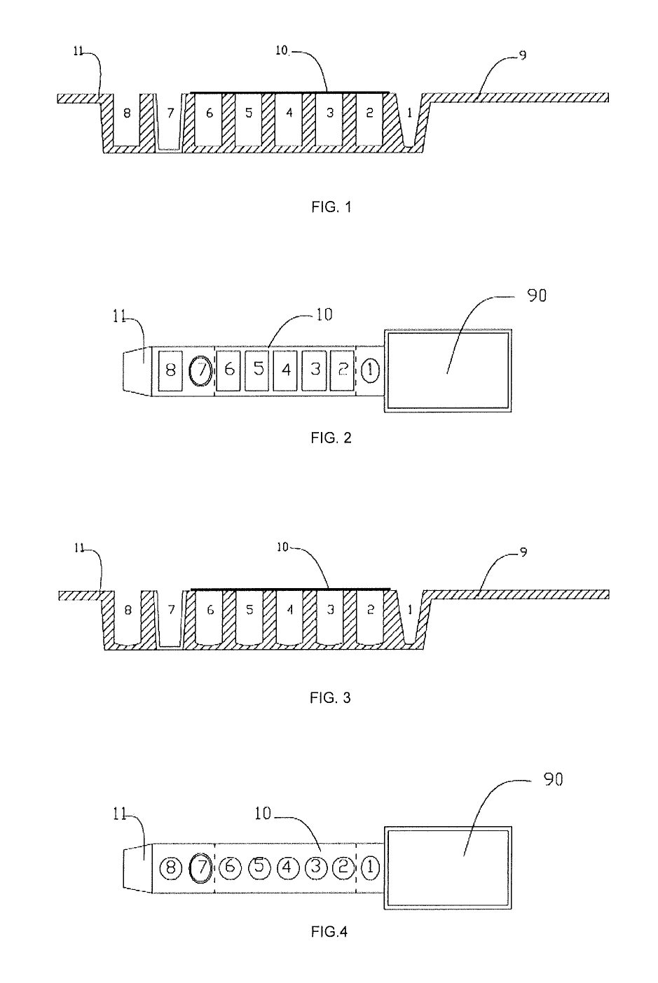

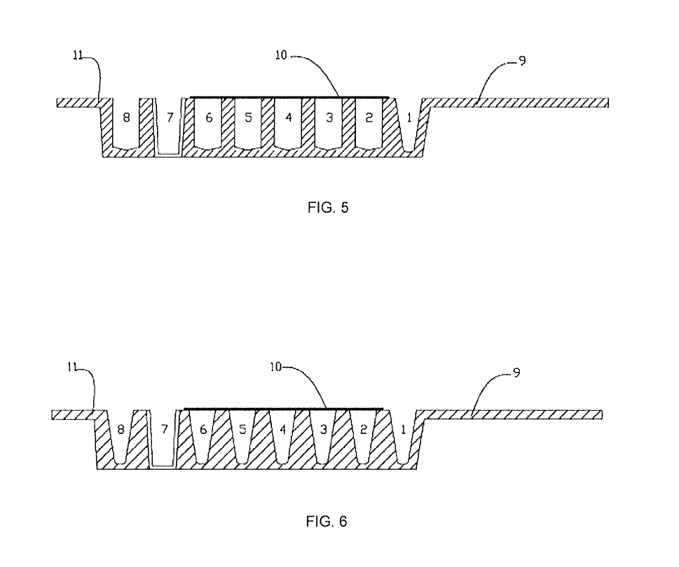

As illustrated in FIGS. 1-6, a reagent device for determining an anti-RA33 antibody IgG by means of (Enzyme-linked Immunosorbent Assay) according to the present invention comprises a substrate 11; wherein the substrate 11 is provided with 1-8 holes (1, 2, 3, 4, 5, 6, 7 and 8), wherein the hole 1 refers to a sample hole used for holding a sample to be detected and the bottom surface of the hole 1 is a ‘V’-shaped groove; the rest holes refer to a reaction hole 7, a dilution hole 8 and reagent holes (2, 3, 4, 5 and 6), wherein the reaction hole 7 is used for receiving detection samples and detection reagents and is served as a reaction hole of an ELASA (Enzyme Linked Immunosorbent Assay) reaction and colorimetric vessel; moreover, the reaction hole is a through hole of a light source/light path permeability; a handle 9 is arranged at one end of the substrate; and a label 90 of an information bar code of the reagent for determining the anti-RA33 antibody IgG is

stuck on the handle. In the example embodiment, the label numbers 2, 3, 4, 5 and 6 refer to the reagent holes; when in use, the reagent holes with the label numbers 2, 3, 4, 5 and 6 have held the reagents required by the detection; moreover, an opening edge of the reaction hole may be square or round and the bottom surface of the reaction hole is a ‘V’-shaped groove; the opening of the reaction hole after holding the reagent or being vacant is sealed by a sealing film 10; the label number 8 refers to the dilution hole which is used for diluting the samples and is not covered by a sealing film.

Other reagents in a kit outside an analytical reagent device comprise: calibrators, quality control materials, buffer washing solution, cleaning solution, disinfectant and distilled water or deionized water.

### Example Embodiment 2 Manufacturing of Reagent Device or Kit

### Indirectly Determining Anti-RA33 antibody IgG

The hole 1 is used as a vessel for holding the sample to be detected; when in use, a solution sample is added for use during detection; the hole 2 is used as an empty hole and the opening of the hole is sealed by a sealing film for standby during detection; and the hole 3 is used as a reagent vessel and the opening of the hole is sealed by a sealing film for use during detection after a sample dilution reagent is added.

The hole 4 is used as a reagent vessel and the opening of the hole is sealed by a sealing film for use during detection after a stop reagent is added; and the hole 5 is used as a reagent vessel and the opening of the hole is sealed by a sealing film for use during detection after an anti-human IgG antibody labeled by HRP (Horse Radish Peroxidase) is added.

The hole 6 is used as a reagent vessel and the opening of the hole is sealed by a sealing film for use during detection after an enzyme reaction substrate is added.

The hole 7 is used as a coating article hole/reaction vessel/colorimetric hole, which is coated by the recombinant RA33 antigen, served as a reaction vessel and filled with the solution sample to be detected, a detection reagent and a washing solution, and finally added with the enzyme reaction substrate for incubation, and then absorbance determination is carried out.

The hole 8 is used as a sample dilution vessel for use during sample dilution.

The handle 9 is stuck with the bar code 90 of the information of the reagent and the analytical device for determining the anti-RA33 antibody IgG, wherein the car code comprises detection item code, detection reagent batch number, reagent validity, qualitative adjusted value/quantitative assay standard curve parameters, ELASA (Enzyme Linked Immunosorbent Assay) reaction type as well as serial numbers of reagents and analytical devices corresponding to each detection.

A plurality of analytical devices are prepared according to the foregoing methods; in addition, corresponding reagents are prepared, which comprise calibrators, quality control materials, buffer washing solution, cleaning solution, disinfectant and distilled water or deionized water, and the like. In this way, complete kit components for determining the anti-RA33 antibody IgG are formed. The reagent devices for determining the anti-RA33 antibody IgG and the ancillary components are filled into an external packaging box of the kit, thus manufacturing the kit for determining the anti-RA33 antibody IgG.

### Example Embodiment 3 Implementing Analytical Operation Flow on Determining Anti-RA33 Antibody IgG on Sample Through Full-Automatic Analyzer

The full-automatic analyzer comprises an analytical device tray which is matched with the shape of the analytical device, wherein the tray is equipped with 30 positions for placing the analytical devices and used for detection and analysis. In addition, a modular integrated mechanical and electronic structure which may implement automatic sample loading, dilution, incubation, washing and reading process is further included. Each position implements quantitative analysis independently; moreover, more than 200 electronic sensor monitoring instruments are operating to guarantee the accuracy of the result. After the instruments are operating, the analytical device tray may automatically rotate to different positions for implementing the steps of sample loading, dilution, incubation, washing and reading.

### (1) Starting up

After an instrument switch is turned on, the instrument may automatically carry out a series of examinations, so as to prepare for the normal operation of the instrument.

(2) Preparation of examination programs

Prepare corresponding solutions as required, comprising buffer washing solution, cleaning solution, disinfectant and distilled water or deionized water, and fill the solutions into corresponding solution tanks after completing the preparation.

(3) Flushing examination

### (4) Preheating

After starting up, the instrument may enable a heating program to adjust temperature to the temperature to be detected.

(5) Connected with a host machine

The instrument may be connected with the host machine through a RS232 serial port, thus submitting a result obtained by normal work of the instrument to a centralized system for processing.

(6) Detection of anti-RA33 antibody IgG
1) Opening a packaging bag from one side with a sealing opening, selecting analytical devices with a quantity as required, deaerating and then sealing the opening of the packaging bag.
2) Examining the substrate in the reagent hole 6 of the analytical device, wherein the substrate shall have no color change; otherwise, the substrate shall be discarded.
3) Respectively adding 50-100 Mu L undiluted samples into the sample holes 1 of each analytical device, wherein for each batch of reagents changed, one analytical device and calibrator thereof shall be selected for calibrating the instrument.
4) Arranging the analytical devices into corresponding analytical device trays in the instruments and carrying out calibration and detection in accordance with an operation instruction (if necessary).
5) Arranging the analytical devices with corresponding quantity into the analytical device trays in accordance with the quantity required for detection, and arranging the analytical devices comprising the calibrators and the quality control materials before a detection position, wherein the instrument may automatically identify the bar codes of the analytical devices, the bar codes of the quality control materials and the bar codes of the calibrators; moreover, a selection line may be located at a ‘sample’ column or ‘detection’ column.
6) Clicking start to operate a detection list, scanning the bar codes of each analytical device, and numbering the quality control materials, the calibrators and the detected samples.
7) Operating the detection list, wherein the instrument may automatically operate according to bar code information; in accordance with the bar codes, the instrument may select correspondingly set standard curves; a program firstly detects the calibrator so as to calibrate a curve preset in the instrument; secondarily, the quality control materials are detected; if a detection result is within a marked scope, then the curve internally arranged is qualified, and may be used for detecting the samples; and finally, a sample detection program is started.
8) Dilution: a filling needle may automatically absorb the samples from the sample hole 1, pierces a hole sealing film 10 to automatically absorb the dilution solution to dilute the samples in the dilution hole 8; after the action is completed, the diluted samples may be moved to the reagent hole 3 by the filling hole and reacted for a period of time set by the program, and then the solution is removed.
9) Washing: the filling needle may absorb a certain quantity of washing solution from a corresponding solution tank to wash the reagent hole 3 for three to five times, and then the solution is removed.
10) The filling needle pierces a reagent hole sealing film 5 to absorb a certain quantity of anti-human IgG antibody labeled by HRP (Horse Radish Peroxidase) to the reagent hole 3 to react for a period of time set by the program, and then the solution is removed.
11) Repeating washing of step (9),
12) The filling needle pierces the sealing film of the reagent hole 6 to absorb a certain quantity of enzyme reaction substrates to the reagent hole 3 to react for a period of time set by the program.
13) The filling needle pierces the sealing film of the reagent hole 4 to absorb a certain quantity of stop solution and fill the solution into the reagent hole 3, and reads OD value in 10 minutes at 450 nm; if a dual wavelength method is selected for determination, then a reference wavelength is 620 nm to 690 nm.
   (7) Detection result

When a detection program operates completely, click data transmission with the host machine, the instrument may automatically send the result obtained by the normal work to the host machine and handle over to external data processing software to analyze and finally generate a report for consulting.

### (8) Shutdown

After the detection is completed, washing circulation shall be enabled before shutdown of the instrument; in this way, residual saline matter from solutions may be avoided from crystallizing in a solution path to prevent the instrument from being damaged or causing invalid detection result; after the washing is completed, the instrument automatically turns off a power supply.

### Example Embodiment 4 Patient Sample Detection Applications, Result Analysis and Detection Quality Control

The operating method and program of the example embodiment 3 and the kit of the example embodiment 2 may be used for quantitatively determining the level of the anti-RA33 antibody IgG in human serum.

The anti-RA33 antibody appeared singly is considered as a marker for RA (Rheumatoid Arthritis); however, the sensitivity of diagnosis thereof is depended on crowd to be detected. The anti-RA33 antibody appears independently without relying on RF (Rheumatoid Factors), and the occurrence rate of the anti-RA33 antibody in negative RF (Rheumatoid Arthritis) is about 45 percent; moreover, the anti-RA33 antibody is significantly important to the early diagnosis of RA (Rheumatoid Arthritis). The anti-RA33 antibody may occur in 70 percent of SLE (Systemic Lupus Erythematosus) patients accompanied with EA (Erosive Arthritis); therefore, the anti-RA33 antibody may be served as a predictive index for development of EA (Erosive Arthritis) in SLE (Systemic Lupus Erythematosus). Therefore, we may carry out clinical diagnosis according to the detection result to primarily judge the disease situation of the patient; moreover, the final diagnosis shall be comprehensively considered by giving references to the clinical manifestation or other diagnosis methods/indexes.

The following is analysis on the detection result:

(1) Reference value (reference scope)

The normal reference value: 0-25 AU/mL; a certain quantity of negative samples (with statistical significances) are detected; an average value of the result plus 3 times of standard deviations (that is, N̅+3SD) is the upper limit of the normal reference value. Each laboratory is suggested to build a normal reference scope thereof according to the actual situation. In order to be convenient for clinical application, we recommend:

Sample value<20 AU/mL Negative

20 AU/mL<sample value<30 AU/mL Doubtful

Sample value>30 AU/mL Positive

(2) Explanations of the detection result

Result explanation: when the sample value is >30 AU/mL, the concentration of the antibody is increased obviously, whether having got RA (Rheumatoid Arthritis) and EA (Erosive Arthritis) shall be diagnosed by giving reference to the clinical manifestation or other diagnosis methods/indexes; when the sample value is <20 AU/mL, the level of the anti-RA33 antibody of an organism is not increased obviously; when 20 AU/mL<the sample value <30 AU/mL, the sample shall be re-detected; if the value is still doubtful, the sample shall be collected again in 2 to 3 weeks for detection.

The results as follows are obtained by repeatedly detecting the positive and negative quality control serums in the detecting process of the example embodiment 3 and detecting the reproducibility of the results:

| | | | |
|---|---|---|---|
| Serial No. | Detection value | Scope | Result |

Positive Quality Control Materials

| | | | |
|---|---|---|---|
| Serial No. 1 | 45.6 | 43 ± 15 | P |
| Serial No. 2 | 49.8 | 43 ± 15 | P |
| Serial No. 3 | 52.3 | 43 ± 15 | P |
| Serial No. 4 | 46.1 | 43 ± 15 | P |
| Serial No. 5 | 49.2 | 43 ± 15 | P |
| Serial No. 6 | 53.6 | 43 ± 15 | P |
| Serial No. 7 | 52.7 | 43 ± 15 | P |
| Serial No. 8 | 49.5 | 43 ± 15 | P |
| Serial No. 9 | 54.0 | 43 ± 15 | P |
| Serial No. 10 | 47.8 | 43 ± 15 | P |

Negative Quality Control Materials

| | | | |
|---|---|---|---|
| Serial No. 1 | 6.8 | <20 | N |
| Serial No. 2 | 7.9 | <20 | N |
| Serial No. 3 | 8.2 | <20 | N |
| Serial No. 4 | 6.7 | <20 | N |
| Serial No. 5 | 6.3 | <20 | N |
| Serial No. 6 | 7.3 | <20 | N |
| Serial No. 7 | 8.0 | <20 | N |
| Serial No. 8 | 7.4 | <20 | N |
| Serial No. 9 | 7.0 | <20 | N |
| Serial No. 10 | 6.8 | <20 | N |

Scope of the determination results is 45.6-53.6 AU/mL, which is in the marked scope (28-58 AU/mL). CV % = 6.0%
The determination results are all less than 20.0 AU/mL, which are negative. CV % = 8.7%

According to the present invention, detection of anti-RA33 antibody IgG on a plurality of samples may be automatically carried out through the same analysis process, which simplifies the detection, reduces the cost, shortens the detection time, is hard to generate cross pollution and is easy to carry out detection operation; moreover, the detection specialty is strong, the sensitivity is high, and the accuracy is good.

What is claimed is:

1. A method for determining an anti-RA33 antibody IgG, wherein the method is implemented by a to a kit consisting of an ELISA (Enzyme-linked Immunosorbent Assay) analytical reagent device and ancillary reagents, wherein the analytical reagent device comprises a substrate provided with 8 holes, and a handle located at one end of the substrate; various specific reagent solutions between the holes of the reagent device are filled and aspirated by a specific analytical instrument to make samples react with the reagents, then a numerical value of solution color after reaction is measured, and finally the measured numerical value is processed to obtain a detection result.

2. The method for determining an anti-RA33 antibody IgG as recited in claim 1, wherein the method comprises the steps as follows: an anti-RA33 antibody IgG to be detected in a sample to be detected is reacted with a recombinant RA33 antigen to form a first immune complex; the first immune complex is reacted with a second antibody labeled by enzyme to form a second immune complex; chromogenic contrast analysis is carried out between the second immune complex formed by reaction and a chromogenic substrate so as to obtain contents of the anti-RA33 antibody IgG to be detected.

3. The method for determining an anti-RA33 antibody IgG as recited in claim 2, wherein the second antibody is an anti-human IgG antibody labeled by HRP (Horse Radish Peroxidase).

4. A reagent device for determining an anti-RA33 antibody IgG, wherein the reagent device is provided with a substrate with 8 holes, a handle located at one end of the substrate, an analytical reagent device used for detecting the anti-RA33 antibody IgG by means of ELISA (Enzyme-linked Immunosorbent Assay) as well as components of corresponding quantities such as ancillary reagent calibrators, quality control materials and buffer washing solutions.

5. The reagent device for determining an anti-RA33 antibody IgG as recited in claim 4, wherein a label of a detection reagent bar code is stuck on the handle located at one end of the substrate; numerical values of the bar code comprise such information as detection item code, detection reagent batch number, reagent validity, qualitative adjusted value/quantitative assay standard curve parameters, ELASA (Enzyme Linked Immunosorbent Assay) reaction type as well as serial numbers of reagents and analytical devices corresponding to each detection.

6. The reagent device for determining an anti-RA33 antibody IgG as recited in claim 4, wherein the holes comprises a reaction hole, a sample hole, a dilution hole and five reagent holes, wherein,
1) The sample hole is used for holding a solution to be detected.
2) The dilution hole is used for diluting samples.
3) The reaction hole is flat in the bottom and has very high light source/light path permeability; when a colourless/blank reagent solution is held, an absorbance value to visible light/ultraviolet light/fluorescent light is approaching to zero; a recombinant RA33 antigen required for detecting the anti-RA33 antibody IgG is coated in the reaction hole; the reaction hole is used for holding detection samples and detection reagents, and generates ELASA (Enzyme Linked Immunosorbent Assay) reaction with the detection samples and the detection reagents; moreover, the reaction hole is a vessel for ELASA (Enzyme Linked Immunosorbent Assay) reaction as well as color display and detection.
4) Each reagent hole is filled with a reagent required for detecting the anti-RA33 antibody IgG by means of ELASA (Enzyme Linked Immunosorbent Assay); after the reagent is filled, an opening edge of a micropore is closed.

7. The reagent device for determining an anti-RA33 antibody IgG as recited in claim 6, wherein the reagents required for detecting the anti-RA33 antibody IgG by means of ELASA (Enzyme Linked Immunosorbent Assay) and held in the reagent holes comprise immune reaction inhibitor/neutralizer/blocker/absorbent, enzyme-conjugate solution, chromogenic substrate solution, chromogenic stop solution, reaction enhancer/accelerator and sample dilution solution required by the ELASA (Enzyme Linked Immunosorbent Assay) for detecting the anti-RA33 antibody IgG.

8. The reagent device for determining an anti-RA33 antibody IgG as recited in claim 6, wherein the section shapes of the sample hole, the reaction hole, the dilution hole and the reagent holes comprise flat shape, V shape or U shape, or any combination between flat shape, V shape and U shape.

9. The method for determining an anti-RA33 antibody IgG as recited in claim 1, wherein the method comprises the steps as follows:
1) Starting up: after an instrument switch is turned on, the instrument may automatically carry out a series of examinations, so as to prepare for the normal operation of the instrument.
2) Preparation of examination programs: prepare corresponding solutions as required, comprising buffer washing solution, cleaning solution, disinfectant and distilled water or deionized water, and fill the solutions into corresponding solution tanks after completing the preparation;
3) Flushing examination:
4) Preheating: after starting up, the instrument may enable a heating program to adjust temperature to the temperature to be detected;
5) Connected with a host machine: the instrument may be connected with the host machine through a RS232 serial port, thus submitting a result obtained by normal work of the instrument to a centralized system for processing;
6) Detection of anti-RA33 antibody IgG: the detection further comprises the steps as follows:
i. Opening a packaging bag from one side with a sealing opening, selecting analytical devices with a quantity as required, deaerating and then sealing the opening of the packaging bag;
ii. Examining the substrate in the reagent hole of the analytical device, wherein the substrate shall have no color change; otherwise, the substrate shall be discarded;
iii. Respectively adding 50-100 Mu L undiluted samples into the sample holes of each analytical device, wherein for each batch of reagents changed, one analytical device and calibrator thereof shall be selected for calibrating the instrument;
iv. Arranging the analytical devices into corresponding analytical device trays in the instruments and carrying out calibration and detection in accordance with an operation instruction.
v. Arranging the analytical devices with corresponding quantity into the analytical device trays in accordance with the quantity required for detection, and arranging the analytical devices comprising the calibrators and the quality control materials before a detection position, wherein the instrument may automatically identify the bar codes of the analytical devices, the bar codes of the quality control materials and the bar codes of the calibrators; moreover, a selection line may be located at a ‘sample’ column or ‘detection’ column,
vi. Clicking start to operate a detection list, scanning the bar codes of each analytical device, and numbering the quality control materials, the calibrators and the detected samples.
vii. Operating the detection list, wherein the instrument may automatically operate according to bar code information; in accordance with the bar codes, the instrument may select correspondingly set standard curves; a program firstly detects the calibrator so as to calibrate a curve preset in the instrument; secondarily, the quality control materials are detected; if a detection result is within a marked scope, then the curve internally arranged is qualified, and may be used for detecting the samples; and finally, a sample detection program is started.
viii. Dilution: a filling needle may automatically absorb the samples from the sample hole, pierces a hole sealing film to automatically absorb the dilution solution to dilute the samples in the dilution hole; after the action is completed, the diluted samples may be moved to the reagent hole by the filling hole and reacted for a period of time set by the program, and then the solution is removed.
ix. Washing: the filling needle may absorb a certain quantity of washing solution from a corresponding solution tank to wash the reagent hole for three to five times, and then the solution is removed.
x. The filling needle pierces a reagent hole sealing film to absorb a certain quantity of anti-human IgG antibody labeled by HRP (Horse Radish Peroxidase) to the reagent hole to react for a period of time set by the program, and then the solution is removed.
xi. Repeating washing of step (9).
xii. The filling needle pierces the sealing film of the reagent hole to absorb a certain quantity of enzyme reaction substrates to the reagent hole to react for a period of time set by the program.
xiii. The filling needle pierces the sealing film of the reagent hole to absorb a certain quantity of stop solution and fill the solution into the reagent hole and reads OD value in 10 minutes at 450 nm; if a dual wavelength method is selected for determination, then a reference wavelength is 620 nm to 690 nm.
7) Detection result: when a detection program operates completely, click data transmission with the host machine, the instrument may automatically send the result obtained by the normal work to the host machine and handle over to external data processing software to analyze and finally generate a report for consulting;
8) Shutdown: after the detection is completed, washing circulation shall be enabled before shutdown of the instrument; in this way, residual saline matter from solutions may be avoided from crystallizing in a solution path to prevent the instrument from being damaged or causing invalid detection result; after the washing is completed, the instrument automatically turns off a power supply.
